(19) United States
(12) Patent Application Publication
(10) Pub. No.: US 2012/0095180 A1
(43) Pub. Date: Apr. 19, 2012

(54) ISOLATION OF A C5-DEPROTONATED IMIDAZOLIUM, A CRYSTALLINE ABNORMAL N-HETEROCYCLIC CARBENE

(75) Inventors: Guy Bertrand, Riverside, CA (US); Bruno Donnadieu, Riverside, CA (US); Eugenia Aldeco-Perez, Riverside, CA (US)

(73) Assignee: The Regents of the University of California Office of Technology Transfer, Oakland, CA (US)

(21) Appl. No.: 13/272,969

(22) Filed: Oct. 13, 2011

Related U.S. Application Data
(60) Provisional application No. 61/393,841, filed on Oct. 15, 2010.

(51) Int. Cl. C08F 36/02; C07C 6/02; C07C 211/00; C07F 5/02; C07D 233/58

(52) U.S. Cl. 526/335; 548/110; 548/343.5; 564/305; 585/645

(57) ABSTRACT

The present invention provides metal-free abnormal N-heterocyclic carbenes, also known as imidazol-5-ylidenes and metal complexes of abnormal N-heterocyclic carbenes. The present invention also provides methods of making metal-free abnormal N-heterocyclic carbenes and metal complexes of abnormal N-heterocyclic carbenes. The present invention also provides methods of using metal-free abnormal N-heterocyclic carbenes and metal complexes of abnormal N-heterocyclic carbenes in catalytic reactions.

### CROSS-REFERENCES TO RELATED APPLICATIONS

This application claims the benefit of Provisional Application Ser. No. 61/393,841, filed Oct. 15, 2010, which is incorporated herein by reference in its entirety.

### STATEMENT AS TO RIGHTS TO INVENTIONS MADE UNDER FEDERALLY SPONSORED RESEARCH OR DEVELOPMENT

This invention was made with Government support under Grant No. GM068825, awarded by the National Institutes of Health. The Government has certain rights in this invention.

### BACKGROUND OF THE INVENTION

Although carbenes were once considered reactive intermediates (R. A. Moss, et al., Reactive Intermediate Chemistry, (Wiley, New York, 2004)), stable carbenes (A. Igau, et al., J. Am. Chem. Soc. 110, 6463 (1988); and D. Bourissou, et al., Chem. Rev. 100, 39 (2000)), and particularly N-heterocyclic carbenes (A. J. Arduengo, et al., J. Am. Chem. Soc. 113, 3122 (1991); A. J. Arduengo, Acc. Chem. Res. 32, 913 (1999); and F. E. Hahn, et al., Angew. Chem. Int. Ed. 48, 950 (2008)), referred herein to as NHCs or imidazol-2-ylidenes, are recognized as versatile ligands for transition metal catalysts and as metal-free organic catalysts (S. E. Denmark, et al., Angew. Chem. Int. Ed. 47, 1560 (2008); N. Marion, et al., Angew. Chem. Int. Ed. 46, 2988 (2007); D. Enders, et al., Chem. Rev. 107, 5606 (2007); N. E. Kamber, et al., Chem. Rev. 107, 5813 (2007)). Previous researchers have prepared metal-free and metal-complexed NHCs, and also metal-complexed abnormal NHCs, referred herein to as aNHCs or alternative N-heterocyclic carbenes or imidazol-5-ylidenes (S. Grundemann, et al., Chem. Commun. (Camb.) 21, 2274 (2001); G. Sini, et al., Inorg. Chem. 41, 602 (2002); and M. Alcarazo et al., J. Am. Chem. Soc. 127, 3290 (2005)). Abnormal NHCs are so named because the first-reported metal-complexed imidazol-5-ylidenes were described as having their imidazole ring bound to the transition metal in the wrong way as a consequence of the carbene ligand bonding to the metal through the C5, and not C2, position of the ring. Since these discoveries, aNHCs have found wide utility as ligands for transition metal complexes (O, Schuster, et al., Chem. Rev. 109, 3445 (2009); M. Albrecht, Chem. Commun. (Camb.) 31, 3601 (2008); and P. l. Arnold, et al., Coord. Chem. Rev. 251, 596 (2007)) and as catalysts in the activation of unreactive bonds such as C—H and H—H(H. Lebel, et al., J. Am. Chem. Soc. 126, 5046 (2004); A. Prades, et al., Organometallics 27, 4254 (2008); and M. Heckenroth, et al., Angew. Chem. Int. Ed. 46, 6293 (2007)).

Despite the progress in preparing metal-free and metal-complexed imidazol-2-ylidenes as well as metal-complexed imidazol-5-ylidenes, previous researchers have not been successful in preparing metal-free imidazol-5-ylidenes. Although Lassaletta et al. were able to isolate metal-free NHCs from deprotonated imidazol[1,5-a]pyridinium salts and also metal-complexed aNHCs when the deprotonation reaction occurred in the presence of [Rh(COD)Cl₂] (M. Alcarazo et al. J. Am. Chem. Soc. 127, 3290 (2005)), Lassaletta et al. were unable to isolate metal-free aNHCs.

What is needed in the art are compounds of, and methods for preparing, isolable and stable metal-free C5-deprotonated imidazolium carbenes, i.e. imidazol-5-ylidenes. Surprisingly, the present invention meets these as well as other needs.

### BRIEF SUMMARY OF THE INVENTION

In a first embodiment, the present invention provides a stable C5-deprotonated imidazolium carbene compound having the structure of Formula I:

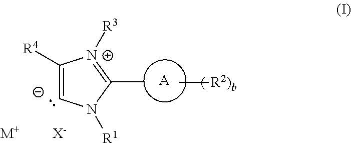

In Formula I, R¹, R³, and R⁴ are independently selected from optionally substituted C₁-C₁₀ alkyl, optionally substituted C₂-C₁₀ alkenyl, optionally substituted C₂-C₁₀ alkynyl, optionally substituted C₃-C₁₀ cycloalkyl, optionally substituted C₃-C₁₀ heterocycloalkyl, optionally substituted aryl, or optionally substituted heteroaryl; R² is, in each instance, independently selected from hydrogen, optionally substituted C₁-C₆ alkyl, optionally substituted C₂-C₆ alkenyl, optionally substituted C₂-C₆ alkynyl, halogen, or hydroxyl; Ring A is aryl or heteroaryl; M is either absent or is an alkali metal cation selected from lithium, sodium, potassium, rubidium, and cesium; X is either absent or is an anion selected from fluoro, chloro, bromo, iodo, trifluoromethanesulfonate, chlorate, acetate, cyanide, thiocynate, oxalate, tetrafluoroborate, nitrate, nitrite, sulfate, sulfite, phosphate, or carboxylate; subscript b is an integer of from 0 to 10.

In a second embodiment, the present invention provides a coordination complex including a metal atom and at least one ligand selected from a carbene having the structure of Formula 1.

In a third embodiment, the present invention provides a reaction mixture including either a carbene of Formula I or a complex of a metal and a carbene of Formula I under conditions sufficient for catalysis to occur, a solvent and an olefin substrate, wherein said olefin substrate is selected to participate in an olefin metathesis reaction.

In a fourth embodiment, the present invention provides a method of making a isolable, stable carbene of Formula I, where the method includes contacting an imidazolium salt in a solvent with a Brönsted base at approximately −78° C.; warming and stirring the mixture of an imidazolium salt in a solvent with a Brönsted base to room temperature; evaporating the solvent under vacuum; and extracting the product with an extracting solvent.

In a fifth embodiment, the present invention provides a method of catalyzing an α-arylation reaction, including combining α-arylation reactants with either a carbene of Formula I or a complex of a metal and a carbene of Formula I under conditions sufficient for catalysis to occur.

In a sixth embodiment, the present invention provides a method of catalyzing a Suzuki coupling reaction, including combining Suzuki coupling reactants with either a carbene of Formula I or a complex of a metal and a carbene of Formula I under conditions sufficient for catalysis to occur.

In a seventh embodiment, the present invention provides a method of catalyzing an amine arylation reaction, including combining amine arylation reactants with either a carbene of Formula I or a complex of a metal and a carbene of Formula I under conditions sufficient for catalysis to occur.

In an eighth embodiment, the present invention provides a method for conducting olefin metathesis, including contacting an olefin substrate with either a carbene of Formula I or a complex of a metal and a carbene of Formula I, under metathesis conditions.

In a ninth embodiment, the present invention provides a method of conducting a reaction selected from a carbon-carbon coupling reaction, a carbon-heteroatom coupling reaction or a 1,2 addition to a multiple bond, said method including contacting suitable substrates selected to undergo at least one of said reactions with either a carbene of Formula I or a complex of a metal and a carbene of Formula I, under suitable reaction conditions.

### BRIEF DESCRIPTION OF THE DRAWINGS

FIG. 1 shows resonance structures for N-heterocyclic carbenes, Compounds A and A′, and abnormal N-heterocyclic carbene, Compounds D and D′, and their corresponding C2 and C5 metal complexes, Compounds B and C, respectively. The synthesis of the metal-complexed aNHC, Compound C1, (S. Grtindemann, et al., Chem. Commun. 2274 (2001)) is shown on the bottom of FIG. 1.

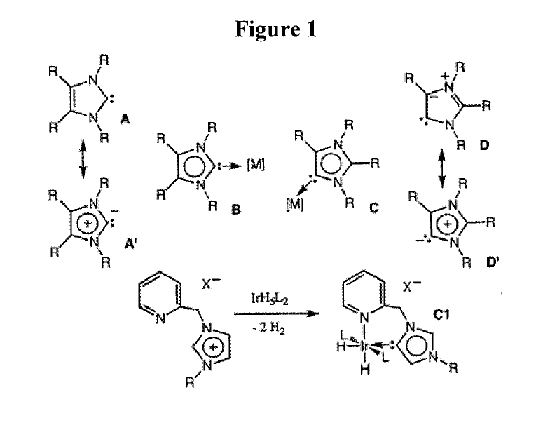

FIG. 2 shows Molecular (ORTEP) views of imidazolium bromide, Compound 2, (left) and an abnormal N-heterocyclic carbene, Compound 6, (right) in the solid state (for clarity H atoms are omitted, except the ring hydrogen). Bond lengths in Compound 2 are the following: C5-N1, 1.368±4 Å; N1-C2, 1.334±4 Å; C2-N3, 1.363+4 Å; N3-C4, 1.408±4 A; C4-C5, 1.355±5 Å. Bond angles in Compound 2 are the following: N1-C5-C4, 108.0±3°; C5-C4-N3, 106.0±3°; C4-N3-C2, 108.8±3°; N3-C2-N1, 106.9±3°; C2-N1-C5, 110.4±3°; 4: C5a-N1a, 1.417±2 Å; N1a-C2a, 1.357±2 Å; C2a-N3a, 1.345±2 Å; N3a-C4a, 1.412±3 Å; C4a-C5a, 1.383±3 Å; N1a-C5a-C4a, 101.03±17°; C5a-C4a-N3a, 111.01±16°; C4a-N3a-C2a, 107.97±15°; N3a-C2a-N1a, 106.25±16°; C2a-N1a-C5a, 113.72±15°.

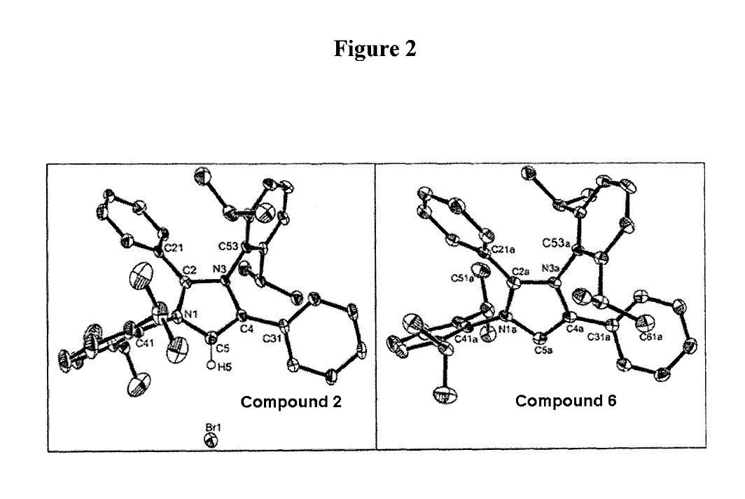

FIG. 3 shows a plot of the two highest-lying occupied molecular orbitals, i.e. HOMO (left) and HOMO-1 (right), of the abnormal N-heterocyclic carbene.

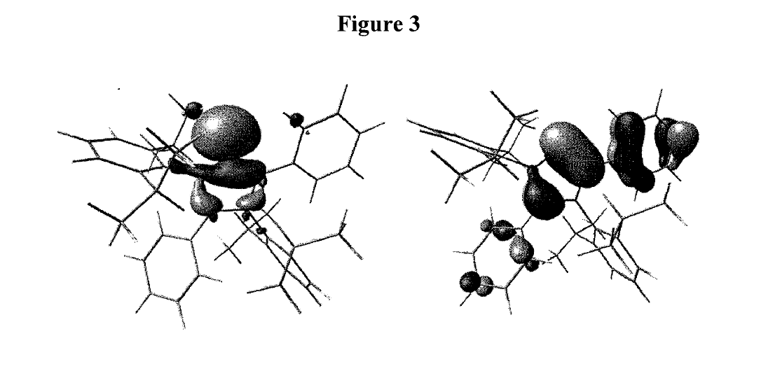

FIG. 4 provides a scheme describing the synthesis of the C2 metal-free N-heterocyclic carbenes and the metal-complexed abnormal N-heterocyclic carbene by Lassaletta et al.

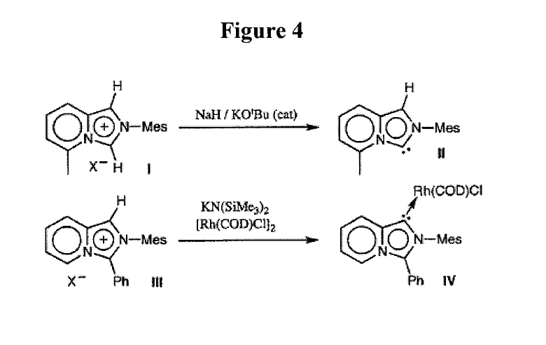

FIG. 5 provides a scheme describing the synthesis of an abnormal N-heterocyclic carbene lithium complex, Compound 4, a rearrangement product of Compound 4, and a free abnormal N-heterocyclic carbene, Compound 6.

### DETAILED DESCRIPTION OF THE INVENTION

### I. General

The present invention provides metal-free abnormal N-heterocyclic carbenes, also known as imidazol-5-ylidenes and metal complexes of abnormal N-heterocyclic carbenes. The present invention also provides methods of making metal-free abnormal N-heterocyclic carbenes and metal complexes of abnormal N-heterocyclic carbenes. The present invention also provides methods of using metal-free abnormal N-heterocyclic carbenes and metal complexes of abnormal N-heterocyclic carbenes in catalytic reactions that include, but are not limited to, an α-arylation reaction, a Suzuki coupling reaction, a carbon-carbon coupling reaction, a carbon-heteroatom coupling reaction, a 1,2 addition to a multiple bond, an amine arylation reaction, and an olefin metathesis reaction.

### II. Definitions

The abbreviations used herein have their conventional meaning within the chemical and biological arts.

Where substituent groups are specified by their conventional chemical formulae, written from left to right, they equally encompass the chemically identical substituents that would result from writing the structure from right to left, e.g., —CH₂O— is equivalent to —OCH₂—.

As used herein, the term “deprotonated” refers to the state of a compound after the removal of a bonded hydrogen atom from the compound. The term “C5-deprotonated” refers a ring-containing compound that is deprotonated at the C5 position on the ring.

As used herein, the term “imidazolium” refers to a compound that includes a 5-membered positively-charged heterocycloalkyl functional group that includes, in the heterocycloalkyl ring, three sp²-hydridized carbon atoms and 2 nitrogen atoms.

As used herein, the term “N-heterocyclic” refers to a heterocycloalkyl functional group that includes at least one nitrogen atom in the heterocycloalkyl ring.

As used herein, the term “C5” refers to the fifth position on a cycloalkyl or heterocycloalkyl ring.

As used herein, the abbreviation “aNHC” refers to abnormal N-heterocyclic carbene.

As used herein, the term “abnormal” refers to an imidazolium-derived compound that is deprotonated at the C5 position on the imidazolium-derived ring.

As used herein, the term “carbene” refers to a class of compounds with a neutral divalent carbon atom with a pair of lone electrons, i.e. a carbon atom that has two lone electrons and is also bonded to two other chemical entities.

As used herein, the term “alkyl” refers to a straight or branched, saturated, aliphatic radical having the number of carbon atoms indicated. For example, C₁-C₆ alkyl includes, but is not limited to, methyl, ethyl, propyl, butyl, pentyl, hexyl, iso-propyl, iso-butyl, sec-butyl, tert-butyl, etc.

As used herein, the term “alkylene” refers to either a straight chain or branched alkylene of 1 to 7 carbon atoms, i.e. a divalent hydrocarbon radical of 1 to 7 carbon atoms; for instance, straight chain alkylene being the bivalent radical of Formula —(CH₂)ₙ—, where n is 1, 2, 3, 4, 5, 6 or 7. Preferably alkylene represents straight chain alkylene of 1 to 4 carbon atoms, e.g. a methylene, ethylene, propylene or butylene chain, or the methylene, ethylene, propylene or butylene chain mono-substituted by C₁-C₃-alkyl (preferably methyl) or disubstituted on the same or different carbon atoms by C₁-C₃-alkyl (preferably methyl), the total number of carbon atoms being up to and including 7. One of skill in the art will appreciate that a single carbon of the alkylene can be divalent, such as in —(HC(CH₂)ₙCH₃)—, wherein n=0-5.

As used herein, the term “heteroalkyl” refers to an alkyl group having from 1 to 3 heteroatoms such as N, O and S. Additional heteroatoms can also be useful, including, but not limited to, B, Al, Si and P. The heteroatoms can also be oxidized, such as, but not limited to, —S(O)— and —S(O)₂—. For example, heteroalkyl can include ethers, thioethers, alkyl-amines and alkyl-thiols.

As used herein, the term “heteroalkylene” refers to a heteroalkyl group, as defined above, linking at least two other groups. The two moieties linked to the heteroalkylene can be linked to the same atom or different atoms of the heteroalkylene.

As used herein, the term “alkenyl” refers to either a straight chain or branched hydrocarbon of 2 to 6 carbon atoms, having at least one double bond. Examples of alkenyl groups include, but are not limited to, vinyl, propenyl, isopropenyl, butenyl, isobutenyl, butadienyl, pentenyl or hexadienyl.

As used herein, the term “alkynyl” refers to either a straight chain or branched hydrocarbon of 2 to 6 carbon atoms, having at least one triple bond. Examples of alkynyl groups include, but are not limited to, acetylenyl, propynyl or butynyl.

As used herein, the term “cycloalkyl” refers to a saturated or partially unsaturated, monocyclic, fused bicyclic or bridged polycyclic ring assembly containing from 3 to 12 ring atoms, or the number of atoms indicated. For example, C₃-C₈ cycloalkyl includes cyclopropyl, cyclobutyl, cyclopentyl, cyclohexyl, cycloheptyl, and cyclooctyl. Cycloalkyl also includes norbornyl and adamantyl.

As used herein, the terms “heterocycloalkyl” and “heterocyclic” refer to a ring system having from 3 ring members to about 20 ring members and from 1 to about 5 heteroatoms such as N, O and S. For example, heterocycle includes, but is not limited to, tetrahydrofuranyl, tetrahydrothiophenyl, morpholino, pyrrolidinyl, pyrrolinyl, imidazolidinyl, imidazolinyl, pyrazolidinyl, pyrazolinyl, piperazinyl, piperidinyl, indolinyl, quinuclidinyl and 1,4-dioxa-8-aza-spiro[4.5]dec-8-yl.

As used herein, the term “aryl” refers to a monocyclic or fused bicyclic, tricyclic or greater, aromatic ring assembly containing 6 to 16 ring carbon atoms. For example, aryl may be phenyl, benzyl or naphthyl, preferably phenyl. “Arylene” means a divalent radical derived from an aryl group. Aryl groups can be mono, di, or tri substituted by one, two or three radicals selected from alkyl, alkoxy, aryl, hydroxy, halogen, cyano, amino, amino alkyl, trifluoromethyl, alkylenedioxy and oxy C₂-C₃ alkylene, or 1 or 2 naphthyl; or 1 or 2 phenanthrenyl.

As used herein, the term “heteroaryl” refers to a monocyclic or fused bicyclic or tricyclic aromatic ring assembly containing 5 to 16 ring atoms, where from 1 to 4 of the ring atoms are a heteroatom each N, O or S. For example, heteroaryl includes pyridyl, indolyl, indazolyl, quinoxalinyl, quinolinyl, isoquinolinyl, benzothienyl, benzofuranyl, furanyl, pyrrolyl, thiazolyl, benzothiazolyl, oxazolyl, isoxazolyl, triazolyl, tetrazolyl, pyrazolyl, imidazolyl, thienyl, or any other radicals substituted, especially mono or di substituted, by e.g. alkyl, nitro or halogen. Pyridyl represents 2, 3, or 4 pyridyl, advantageously 2 or 3 pyridyl. Thienyl represents 2 or 3 thienyl. Quinolinyl represents preferably 2, 3, or 4 quinolinyl. Isoquinolinyl represents preferably 1, 3, or 4 isoquinolinyl. Benzopyranyl, benzothiopyranyl represents preferably 3 benzopyranyl or 3 benzothiopyranyl, respectively. Thiazolyl represents preferably 2 or 4 thiazolyl, and most preferred, 4 thiazolyl. Triazolyl is preferably 1, 2, or 5 (1,2,4 triazolyl). Tetrazolyl is preferably 5 tetrazolyl.

Preferably, heteroaryl is pyridyl, indolyl, quinolinyl, pyrrolyl, thiazolyl, isoxazolyl, triazolyl, tetrazolyl, pyrazolyl, imidazolyl, thienyl, furanyl, benzothiazolyl, benzofuranyl, isoquinolinyl, benzothienyl, oxazolyl, indazolyl, or any of the radicals substituted, especially mono or di substituted.

Substituents for the aryl and heteroaryl groups are varied and are selected from: halogen, OR′, OC(O)R′, NR′R″, SR′, R′, CN, NO₂, CO₂R′, CONR′R″, C(O)R′, OC(O)NR′R″, NR″C(O)R′, NR″C(O)₂R′, NR′C(O)NR″R″′, NH C(NH₂)═NH, NR′C(NH₂)═NH, NH C(NH₂)═NR′, S(O)R′, S(O)₂R′, S(O)₂NR′R″, N₃, CH(Ph)₂, perfluoro(C₁-C₄)alkoxy, and perfluoro(C₁-C₄)alkyl, in a number ranging from zero to the total number of open valences on the aromatic ring system; and where R′, R″ and R″′ are independently selected from hydrogen, C₁-C₈ alkyl and heteroalkyl, unsubstituted aryl and heteroaryl, (unsubstituted aryl) (C₁-C₄)alkyl, and (unsubstituted aryl)oxy(C₁-C₄)alkyl.

As used herein, the terms “halo” or “halogen,” by themselves or as part of another substituent, mean, unless otherwise stated, a fluorine, chlorine, bromine, or iodine atom.

As used herein, the term “hydroxyl” refers to the radical having the formula OH.

As used herein, the phrase “alkali metal” refers to the elements and cations of group 1 of the periodic table and include lithium, sodium, potassium, rubidium, cesium, and francium.

As used herein, the term “cation” refers to a positively-charged atom. For example, cation includes, but is not limited to, Li⁺, Na⁺, K⁺, Rb⁺, and Cs⁺.

As used herein, the term “anion” refers to a negatively-charged atom. For example, anion includes, but is not limited to, F⁻, Cl⁻, Br⁻, I⁻.

Each of the above terms (e.g., “alkyl,” “heteroalkyl,” “aryl” and “heteroaryl”), when indicated as “substituted” or “optionally substituted,” are meant to include both substituted and unsubstituted forms of the indicated radical.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R″′)═NR″″, —NR—C(NR′R″)═NR″′, —S(O)R′, —S(O)₂R′, —S(O)₂NR′R″, —NR(SO₂)R′, —CN and —NO₂ in a number ranging from zero to (2 m′+1), where m′ is the total number of carbon atoms in such radical. R′, R″, R″′ and R″″ are each independently selected from hydrogen, C₁-C₈ alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R″′ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “substituted alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

As used herein, the term “hydrate” refers to a compound that is complexed to at least one water molecule. The compounds of the present invention can be complexed with from 1 to 10 water molecules.

As used herein, the term “ligand” refers to an ion or a molecule that bonds to a central metal atom.

As used herein, the phrase “coordinating metal ion” refers to a metal such as, but not limited to, an alkali metal, an alkaline earth metal, a transition metal such as, but not limited to, Co, Rh, Ir, Ni, Pd, Pt, Cu, Ag, and Au, which can bond to a ligand such as, but not limited to, the free-carbenes of the present application.

As used herein, the phrase “coordination complex” refers to a complex that includes a coordinating metal ion and a ligand.

As used herein, the phrase “Brönsted base” refers to a compound that is capable of bonding to and accepting a hydrogen cation.

As used herein, the phrase “room temperature” refers to the temperature of a laboratory under ambient conditions. For example, room temperature includes the range of from about 18° C. to about 28° C. Preferably, room temperature includes the range of from about 20° to about 25° C.

As used herein, the term “isomers” refers to compounds having the same number and kind of atoms, and hence the same molecular weight, but differing in respect to the structural arrangement or configuration of the atoms.

As used herein, the term “tautomer,” refers to one of two or more structural isomers which exist in equilibrium and which are readily converted from one isomeric form to another.

As used herein, the terms “a,” “an,” or “a(n)”, when used in reference to a group of substituents or “substituent group” herein, mean at least one. For example, where a compound is substituted with “an” alkyl or aryl, the compound is optionally substituted with at least one alkyl and/or at least one aryl, wherein each alkyl and/or aryl is optionally different. In another example, where a compound is substituted with “a” substituent group, the compound is substituted with at least one substituent group, wherein each substituent group is optionally different.

Description of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, or neutral conditions.

### III. Compounds

The present invention provides stable C5-deprotonated imidazolium carbene compounds and metal complexes of C5-deprotonated imidazolium carbene compounds.

In some embodiment, the present invention provides a stable C5-deprotonated imidazolium carbene compound having the structure of Formula I:

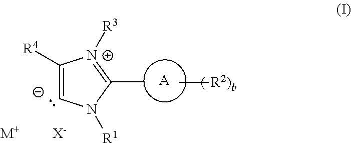

In Formula I, R¹, R³, and R⁴ are independently selected from optionally substituted C₁-C₁₀ alkyl, optionally substituted C₂-C₁₀ alkenyl, optionally substituted C₂-C₁₀ alkynyl, optionally substituted C₃-C₁₀ cycloalkyl, optionally substituted C₃-C₁₀ heterocycloalkyl, optionally substituted aryl, or optionally substituted heteroaryl; R² is, in each instance, independently selected from hydrogen, optionally substituted C₁-C₆ alkyl, optionally substituted C₂-C₆ alkenyl, optionally substituted C₂-C₆ alkynyl, halogen, or hydroxyl; Ring A is aryl or heteroaryl; M is either absent or is an alkali metal cation selected from lithium, sodium, potassium, rubidium, and cesium; X is either absent or is an anion selected from fluoro, chloro, bromo, iodo, trifluoromethanesulfonate, chlorate, acetate, cyanide, thiocynate, oxalate, tetrafluoroborate, nitrate, nitrite, sulfate, sulfite, phosphate, or carboxylate; subscript b is an integer of from 0 to 10. In some embodiments, X is fluoro or chloro. In some other embodiments, M is Na or K. In some embodiments, M is Li.

In some other embodiments, the present invention provides a compound of Formula I, wherein ring A is selected from phenyl, benzyl, naphthyl, phenanthrenyl, anthracyl, pyridyl, indolyl, indazolyl, quinoxalinyl, quinolinyl, isoquinolinyl, benzothienyl, benzofuranyl, furanyl, pyrrolyl, thiazolyl, benzothiazolyl, oxazolyl, isoxazolyl, triazolyl, tetrazolyl, pyrazolyl, imidazolyl, or thienyl. In other embodiments, the present invention provides a compound of Formula A, wherein R¹, R³, and R⁴ are each optionally substituted phenyl.

In some embodiments, the present invention provides a compound having the structure of Formula II:

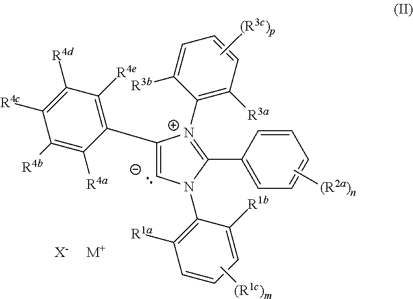

In Formula II, R^{1a}, R^{1b}, R^{3a}, and R^{3b} are independently selected from hydrogen and optionally substituted C₁-C₁₀ alkyl; R^{1c}, R^{2a}, R^{3c} are, in each instance, independently selected from hydrogen, optionally substituted C₁-C₆ alkyl, optionally substituted C₂-C₆ alkenyl, optionally substituted C₂-C₆ alkynyl, halogen, and hydroxyl; R^{4a}, R^{4b}, R^{4c}, R^{4d}, and R^{4e} are independently selected from hydrogen, optionally substituted C₁-C₆ alkyl, optionally substituted C₂-C₆ alkenyl, optionally substituted C₂-C₆ alkynyl, halogen, or hydroxyl; M is either absent or is an alkali metal cation selected from lithium, sodium, potassium, rubidium, and cesium; X is either absent or is a halogen anion selected from fluoro, chloro, bromo, or iodo; subscripts m and p are independently integers of from 0 to 3; the subscript n is an integer of from 0 to 5; Also included are the salts of the carbenes of Formula II. In some embodiments, X is fluoro or chloro. In some other embodiments, M is Na or K. In some embodiments, M is Li. In some other embodiments, R^{1a}, R^{1b}, R^{3a} and R^{3b} are independently selected from an optionally substituted C₂-C₆ alkyl. In some embodiments, R^{1a}, R^{1b}, R^{3a}, and R^{3b} are independently selected an optionally substituted and optionally branched C₃-C₅ alkyl. In some other embodiments, R^{1a}, R^{1b}, R^{3a}, and R^{3b} are each isopropyl.

### IV. Transition Metal Complexes

In some embodiments, the present invention provides metal complexes, including at least one ligand selected from the carbene compounds of Formula I, that are useful as catalysts in a variety of organic reactions. One of skill in the art will appreciate that such complexes can employ a number of metals, including, but not limited to, transition metals, and have a variety of geometries (e.g., trigonal, square planar, trigonal bipyramidal and the like) depending on the nature of the metal and its oxidation state and other factors including, for example, additional ligands.

In some other embodiments, the present invention provides a coordination complex including a metal atom and at least one ligand selected from a carbene compound of Formula I.

In some embodiments, the present invention provides a coordination complex including a metal atom and at least one ligand selected from a carbene compound of Formula I, wherein the metal atom is selected from Li, Na, K, Rb, Cs, Be, Mg, Ca, Sr, Ba, Ra, Sc, Y, La, Ti, Zr, Hf, V, Nb, Ta, Cr, Mo, W, Mn, Tc, Re, Fe, Ru, Os, Co, Rh, Ir, Ni, Pd, Pt, Cu, Ag, Au, Zn, Cd, Hg, Al, Ga, In, Tl, Ge, Sn, Pb, Sb, Bi, or Po. In some embodiments, the metal atom is selected from Ir, Pd, Rh Ru, or Au. In some other embodiments, the coordination complex further includes at least one ligand selected from halide, pseudohalide, tetraphenylborate, perhalogenated tetraphenylborate, tetrahaloborate, hexahalophosphate, hexahaloantimonate, trihalomethanesulfonate, alkoxide, carboxylate, tetrahaloaluminate, tetracarbonylcobaltate, hexahaloferrate(III), tetrahaloferrate(III), tetrahalopalladate(II), alkylsulfonate, arylsulfonate, perchlorate, cyanide, thiocyanate, cyanate, isocyanate, isothiocyanate, amines, imines, phosphines, phosphites, carbonyl compounds, alkenyl compounds, allyl compounds, carboxyl compounds, nitriles, alcohols, ethers, thiols or thioethers. In some embodiments, the coordination complex includes gold; a carbene selected from Formula I or Formula II; and optionally a member selected from bent-allenes, phosphines, sulfonated phosphines, phosphites, phosphinites, phosphonites, arsines, stibines, ethers, ammonia, amines, amides, sulfoxides, carbonyls, nitrosyls, pyridines and thioethers.

In general, any transition metal (e.g., a metal having d electrons) can be used to form the complexes/catalysts of the present invention. For example, suitable transition metals are those selected from one of Groups 3-12 of the periodic table or from the lanthanide series. Preferably, the metal will be selected from Groups 5-12 and even more preferably Groups 7-11. For example, suitable metals include platinum, palladium, iron, nickel, iridium, ruthenium and rhodium. The particular form of the metal to be used in the reaction is selected to provide, under the reaction conditions, metal centers which are coordinately unsaturated and not in their highest oxidation state.

To further illustrate, suitable transition metal complexes and catalysts include soluble or insoluble complexes of platinum, palladium, iridium, iron, rhodium, ruthenium and nickel. Palladium, rhodium, iridium, ruthenium and nickel are particularly preferred and palladium is most preferred.

The transition metal complexes of the present invention can include additional ligands as required to obtain a stable complex. The additional ligands can be neutral ligands, anionic ligands and/or electron-donating ligands. The ligand can be added to the reaction mixture in the form of a metal complex, or added as a separate reagent relative to the addition of the metal.

Anionic ligands suitable as additional ligands are preferably halide, pseudohalide, tetraphenylborate, perhalogenated tetraphenylborate, tetrahaloborate, hexahalophosphate, hexahaloantimonate, trihalomethanesulfonate, alkoxide, carboxylate, tetrahaloaluminate, tetracarbonylcobaltate, hexahaloferrate(III), tetrahaloferrate(III) or/and tetrahalopalladate(II). Preferably, an anionic ligand is selected from halide, pseudohalide, tetraphenylborate, perfluorinated tetraphenylborate, tetrafluoroborate, hexafluorophosphate, hexafluoroantimonate, trifluoromethanesulfonate, alkoxide, carboxylate, tetrachloroaluminate, tetracarbonylcobaltate, hexafluoroferrate (III), tetrachloroferrate(III) or/and tetrachloropalladate(II). Preferred pseudohalides are cyanide, thiocyanate, cyanate, isocyanate and isothiocyanate. Neutral or electron-donor ligands suitable as additional ligands can be, for example, amines, imines, phosphines, phosphites, carbonyl compounds, alkenyl compounds (e.g., allyl compounds), carboxyl compounds, nitriles, alcohols, ethers, thiols or thioethers. Still other suitable ligands can be carbene ligands such as the diaminocarbene ligands (e.g., NHCs).

While the present invention describes a variety of transition metal complexes useful in catalyzing organic reactions, one of skill in the art will appreciate that many of the complexes can be formed in situ. Accordingly, ligands (either carbene ligands or additional ligands) can be added to a reaction solution as a separate compound, or can be complexed to the metal center to form a metal-ligand complex prior to its introduction into the reaction solution. The additional ligands are typically compounds added to the reaction solution which can bind to the catalytic metal center. In some preferred embodiments, the additional ligand is a chelating ligand. While the additional ligands can provide stability to the catalytic transition metal complex, they may also suppress unwanted side reactions as well as enhance the rate and efficiency of the desired processes. Still further, in some embodiments, the additional ligands can prevent precipitation of the catalytic transition metal. Although the present invention does not require the formation of a metal-additional ligand complex, such complexes have been shown to be consistent with the postulate that they are intermediates in these reactions and it has been observed the selection of the additional ligand has an affect on the course of the reaction.

In related embodiments, the present invention provides metal complexes, of the type described above, in which the carbene ligand has a pendent functionalized side chain (e.g., aminoalkyl, mercaptoalkyl, acyloxyalkyl and the like) in which the functional group acts as a ligand to provide a bidentate ligand feature. In still other embodiments, the carbene ligand forms a metal complex with bidentate ligands that are not tethered to the cyclic carbene moiety.

In some embodiments, the present invention provides a reaction mixture including a coordination complex including a metal atom and at least one ligand selected from a carbene compound of Formula I under conditions sufficient for catalysis to occur, a solvent and an olefin substrate, wherein said olefin substrate is selected to participate in an olefin metathesis reaction. In some other embodiments, the olefin substrate is selected as a substrate for ring closing metathesis. In some embodiments, the olefin substrate is selected as a substrate for ring opening polymerization metathesis. In some other embodiments, the olefin substrate is selected as a substrate for cross metathesis. In some embodiments, the olefin substrate is selected as a substrate for acyclic diene polymerization metathesis.

### V. Methods of Making the Compounds and Complexes of the Present Invention

The present invention provides methods of making and using the compounds having the structure of Formulas I and II. See E. Aldeco-Perez, “Isolation of a C5-Deprotonated Imidazolium, a Crystalline ‘Abnormal’ N-Heterocyclic Carbene, Science, 326, 556, (2009), which is incorporated herein by reference in its entirety.

In some embodiments, the present invention provides a method of making a isolable, stable carbene compound of Formula I, the method including contacting an imidazolium salt in a solvent with a Brönsted base at a temperature of from about −20 to −100° C.; warming and stirring the mixture of an imidazolium salt in a solvent with a Brönsted base to room temperature; removing the solvent under vacuum; and extracting the product with an extracting solvent. In some embodiments, the imidazolium salt has the structure of Formula III:

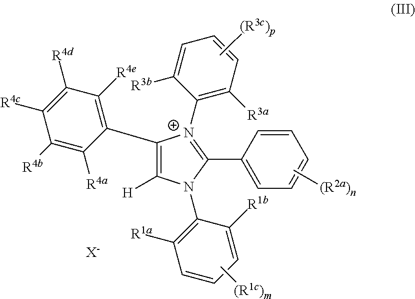

In Formula III, R^{1a}, R^{1b}, R^{3a}, and R^{3b}, are independently selected from hydrogen and optionally substituted C₁-C₁₀ alkyl; R^{1c}, R^{2a}, R^{3c} are in each instance, independently selected from hydrogen, optionally substituted C₁-C₆ alkyl, optionally substituted C₂-C₆ alkenyl, optionally substituted C₂-C₆ alkynyl, halogen, or hydroxyl; R^{4a}, R^{4b}, R^{4c}, R^{4d}, and R^{4e} are independently selected from hydrogen, optionally substituted C₁-C₆ alkyl, optionally substituted C₂-C₆ alkenyl, optionally substituted C₂-C₆ alkynyl, halogen, or hydroxyl; X is either absent or is a halogen anion selected from fluoro, chloro, bromo, or iodo; subscripts m and p are independently integers of from 0 to 3; the subscript n is an integer of from 0 to 5. In some embodiments, the Brönsted base is selected from lithium diisopropylamide, potassium bis(trimethylsilyl)amide, or potassium hexamethyldisilazide. In some other embodiments, the solvent is selected from tetrahydrofuran. In other embodiments, the Brönsted base is a large bulky base, e.g. potassium t-butoxide. In some other embodiments, the contacting of an imidazolium salt in a solvent with a Brönsted base occurs at a temperature of approximately −78° C. In some embodiments, the extracting solvent is selected from hexane, diethyl ether, or combinations thereof.

Brönsted bases suitable for use with the present invention include, but are not limited to, those bases that include cation selected from rows 3, 4, and 5 of the periodic table. In other embodiments, the Brönsted base includes, but is not limited to, a base that includes a cation selected from rows 3 and 4 of the periodic table. In some other embodiments, the cation component to a Brönsted base is potassium, sodium, magnesium, cesium, calcium or barium. In other embodiments, the cation associated with a Brönsted base is sodium or potassium. In some other embodiments, the cation is potassium. Examples of specific Brönsted bases that are suitable for use with the present invention include, but are not limited to, potassium bis(trimethylsilyl)amide, sodium bis(trimethylsilyl)amide, potassium hydride, sodium hydride, sodium and potassium alkoxides (e.g., sodium methoxide, sodium tert-butoxide, potassium tert-butoxide), sodium and potassium aryloxides and derivatives thereof. In other embodiments, the Brönsted bases suitable for use with the present invention include, but are not limited to, potassium bis(trimethylsilyl)amide, sodium bis(trimethylsilyl)amide, potassium hydride and sodium hydride.

### VI. Catalytic Reactions Suitable for Use with the Compounds and Complexes of the Present Invention

As noted above, the compounds and complexes of the present invention are useful in catalyzing a variety of synthetic organic reactions including amine arylation reactions, Suzuki coupling reactions (aryl-aryl or aryl-alkyl coupling reactions), and α-arylation reactions. Still other reactions that can benefit from the above-noted compounds and complexes include, for example, hydroformylation (of alkenes and alkynes), hydrosilylation (of alkenes, alkynes, ketones and aldehydes), metathesis (olefin (RC, CM, ROM, ROMp) ene-yne), carbonylation, hydroarylation and hydroamination.

The reactions of the present invention can be performed under a wide range of conditions, and the solvents and temperature ranges recited herein should not be considered limiting. In general, it is desirable for the reactions to be run using mild conditions which will not adversely affect the reactants, the catalyst, or the product. For example, the reaction temperature influences the speed of the reaction, as well as the stability of the reactants and catalyst. The reactions will typically be run at temperatures in the range of 25° C. to 300° C., more preferably in the range 25° C. to 150° C.

Additionally, the reactions are generally carried out in a liquid reaction medium, but in some instances can be run without addition of solvent. For those reactions conducted in solvent, an inert solvent is preferred, particularly one in which the reaction ingredients, including the catalyst, are substantially soluble. Suitable solvents include ethers such as diethyl ether, 1,2-dimethoxyethane, diglyme, t-butyl methyl ether, tetrahydrofuran and the like; halogenated solvents such as chloroform, dichloromethane, dichloroethane, chlorobenzene, and the like; aliphatic or aromatic hydrocarbon solvents such as benzene, xylene, toluene, hexane, pentane and the like; esters and ketones such as ethyl acetate, acetone, and 2-butanone; polar aprotic solvents such as acetonitrile, dimethylsulfoxide, dimethylformamide and the like; or combinations of two or more solvents.

In some embodiments, reactions utilizing the catalytic complexes of the present invention can be run in a biphasic mixture of solvents, in an emulsion or suspension, or in a lipid vesicle or bilayer. In certain embodiments, the catalyzed reactions can be run in the solid phase with one of the reactants tethered or anchored to a solid support.

In certain embodiments it is preferable to perform the reactions under an inert atmosphere of a gas such as nitrogen or argon.

The reaction processes of the present invention can be conducted in continuous, semi-continuous or batch fashion and may involve a liquid recycle operation as desired. The processes of this invention are preferably conducted in batch fashion. Likewise, the manner or order of addition of the reaction ingredients, catalyst and solvent are also not generally critical to the success of the reaction, and may be accomplished in any conventional fashion.

The reaction can be conducted in a single reaction zone or in a plurality of reaction zones, in series or in parallel or it may be conducted batchwise or continuously in an elongated tubular zone or series of such zones. The materials of construction employed should be inert to the starting materials during the reaction and the fabrication of the equipment should be able to withstand the reaction temperatures and pressures. Means to introduce and/or adjust the quantity of starting materials or ingredients introduced batchwise or continuously into the reaction zone during the course of the reaction can be conveniently utilized in the processes especially to maintain the desired molar ratio of the starting materials. The reaction steps may be effected by the incremental addition of one of the starting materials to the other. Also, the reaction steps can be combined by the joint addition of the starting materials to the metal catalyst. When complete conversion is not desired or not obtainable, the starting materials can be separated from the product and then recycled back into the reaction zone.

The processes may be conducted in either glass lined, stainless steel or similar type reaction equipment. The reaction zone may be fitted with one or more internal and/or external heat exchanger(s) in order to control undue temperature fluctuations, or to prevent any possible “runaway” reaction temperatures.

Furthermore, one or more of the reactants can be immobilized or incorporated into a polymer or other insoluble matrix by, for example, derivativation with one or more of substituents of the aryl group.

In some embodiments, the present invention provide a method of catalyzing an α-arylation reaction, including combining α-arylation reactants with either a carbene compound of Formula I or a coordination complex including a metal atom and at least one ligand selected from a carbene compound of Formula I, under conditions sufficient for catalysis to occur.

In some other embodiments, the present invention provide a method of catalyzing a Suzuki coupling reaction, including combining Suzuki coupling reactants with either a carbene compound of Formula I or a coordination complex including a metal atom and at least one ligand selected from a carbene compound of Formula I under conditions sufficient for catalysis to occur.

In some embodiments, the present invention provides a method of catalyzing an amine arylation reaction, including combining amine arylation reactants with either a carbene compound of Formula I or a coordination complex including a metal atom and at least one ligand selected from a carbene compound of Formula I under conditions sufficient for catalysis to occur

In some other embodiments, the present invention provides a method for conducting olefin metathesis, including contacting an olefin substrate with either a carbene compound of Formula I or a coordination complex including a metal atom and at least one ligand selected from a carbene compound of Formula I, under metathesis conditions. In some embodiments, the olefin substrate is selected as a substrate for ring closing metathesis. In some other embodiments, the olefin substrate is selected as a substrate for ring opening polymerization metathesis. In some embodiments, the olefin substrate is selected as a substrate for cross metathesis. In some other embodiments, the olefin substrate is selected as a substrate for acyclic diene polymerization metathesis.

In some embodiments, the present invention provides a method of conducting a reaction selected from a carbon-carbon coupling reaction, a carbon-heteroatom coupling reaction or a 1,2 addition to a multiple bond, said method including contacting suitable substrates selected to undergo at least one of said reactions with either a carbene compound of Formula I or a coordination complex including a metal atom and at least one ligand selected from a carbene compound of Formula I, under suitable reaction conditions. In some embodiments, the reaction is a carbon-carbon coupling reaction and said suitable conditions include an organic solvent and a temperature of from, −30° C. to 190° C. In some embodiments, the reaction is a carbon-heteroatom coupling reaction and said suitable conditions include an organic solvent and a temperature of from −30° C. to 190° C. In some embodiments, the reaction is a 1,2-addition to a multiple bond and said suitable conditions include an organic solvent and a temperature of from −30° C. to 190° C.

### VII. Examples

All synthetic experiments were carried out under dry argon using standard Schlenk or dry box techniques. Solvents were dried by standard methods and distilled under argon. ¹H and ¹³C-NMR spectra were recorded on Varian Inova 400, 500 and Brucker 300 spectrometers at a temperature of 25° C. and referenced to the residual ¹H, and ¹³C signals of the solvents. NMR multiplicities are abbreviated as follows: s=singlet, d=doublet, t=triplet, sept.=septet, m=multiplet, br=broad signal. Coupling constants J are given in Hz. Mass spectra were performed at the UC Riverside Mass Spectrometry Laboratory. Melting points were measured with a Büchi melting point apparatus system.

### Example 1

### Synthesis of N,N′ bis(2,6-diisopropylphenyl)-N-(2-oxo-2-phenylethyl)benzimidamide, having the following structure

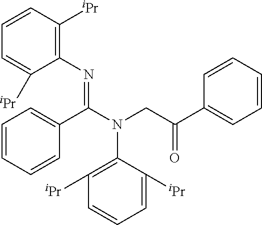

An isopropanol solution (30 mL) of N,N′-bis(2,6-iisopropylphenyl)benzimidamide (10 g, 21 mmol), 2-bromoacetophenone (4.5 g, 23 mmol) and potassium bicarbonate (5 g, 50 mmol) was heated under reflux for 24 hours. Filtration of the potassium bicarbonate and evaporation of the solvent gave an oil. After adding hexane (20 mL), the solution was heated under reflux for 5 minutes, and a white precipitate was obtained by cooling the solution down to 0° C. Recrystallization from ethanol afforded the title compound as colorless crystals (7.6 g, 60% yield). M.P. 142-143° C.; ¹H NMR (CDCl₃, 25° C., 300 MHz): 8.05 (d, J=8 Hz, 2H, CHₐᵣ), 7.55-7.14 (m, 4H, CHₐᵣ), 7.07-6.79 (m, 10H, 5.04 (s, 2H, CH₂), 4.59 (sept, J=6.7 Hz, 2H, CHCH₃), 3.16 (sept, J=6.7 Hz, 2H, CHCH₃), 1.26 (d, J=7 Hz, 12H, CH₃), 1.06 (d, J=7 Hz, 6H, CH₃, 0.97 (d, J=7 Hz, 6H, CH₃); ¹³C NMR (CDCl₃, 25° C., 75 MHz): 193.2 (CO), 156.4 (C═N), 147.8 (Cₐᵣ), 144.8 (Cₐᵣ), 140.6 (Cₐᵣ), 138.7 (Cₐᵣ), 136.7 (Cₐᵣ), 132.9 (CHₐᵣ)′ 132.4 (Cₐᵣ), 128.9 (CHₐᵣ)′ 128.7 (CHₐᵣ)′ 128.5 (CHₐᵣ)′ 127.9 (CHₐᵣ)′ 127.1 (CHₐᵣ)′ 124.3 (CHₐᵣ)′ 122.5 (CHₐᵣ)′ 122.1 (CHₐᵣ), 58.2 (CH₂), 28.3 (CHCH₃), 28.0 (CHCH₃), 26.5 (CH₃), 25.0 (CH₃), 23.0 (CH₃), 22.3 (CH₃); HRMS: m/z calculated for C₃₉H₄₇N₂₀ 559.3683. found 559.3694. As used herein, the acronym “M.P.” refers to the melting point. As used herein, the ackronym “HRMS” refers to high resolution mass spectroscopy.

### Example 2

### Synthesis of N,N′-Bis(1,3-bis(2,6-diisopropylphenyl)-2,4,-diphenylimidazolium (Compound 1) (BF4−)

HBF₄.OEt₂ (1.4 mL, 10.2 mmol) was added dropwise at 0° C. to a suspension of N,N′ bis(2,6-diisopropylphenyl)-N-(2-oxo-2-phenylethyl)benzimidamide (1.75 g, 3.1 mmol) in acetic anhydride (2 ml). The mixture was warmed to room temperature and stirred overnight. Water (20 mL) and then CH₂Cl₂ (20 mL) were added at 0° C. The organic layer was separated, washed with water (3×20 mL), and dried with anhydrous MgSO₄. After evaporation of the solvent under vacuum, the residue was washed with Et₂O, giving Compound 1 (BF₄) as a white solid. Recrystallization in CHCl₃:hexane afforded colorless crystals (1.75 g, 90% yield). M.P. 242-243° C.; ¹H NMR (CDCl₃, 25° C., 300 MHz): 8.31 (s, 1 H, CH_{imidazolium}), 7.66-7.41 (m, 2H, CHₐᵣ), 7.40-7.23 (m, 12H, CH.), 6.95 (d, J=9 Hz, 2H, CHₐᵣ), 2.56-2.46 (m, 4H, CHCH₃), 1.35 (d, J=6.6Hz, 6H, CH₃), 1.01 (d, J=6.6Hz, 6H, CH₃), 0.86 (br, 12H, CH₃); ¹³C NMR (CDCl₃, 25° C., 100 MHz): 145.5 (C), 145.2 (C), 144.8 (C), 137.4 (C), 133.1 (CHₐᵣ), 133.0 (CHₐᵣ)′ 132.6 (CHₐᵣ), 131.0 (CHₐᵣ)′ 130.3 (C), 129.9 (CHₐᵣ)′ 129.7 (CHₐᵣ), 129.4 (CHₐᵣ)′ 128.8 (CHₐᵣ), 128.8 (C), 126.3 (CHₐᵣ)′ 125.6 (CHₐᵣ)′ 124.5 (C), 123.2 (CH_{imidazolium}), 121.0 (C), 29.4 (CHCH₃), 29.1 (CHCH₃), 25.3 (CH₃), 23.7 (CH₃), 23.3 (CH₃), 22.5 (CH₃); HRMS: m/z calculated for ˜9H₄₅N2 541.3577. found M 541.3578.

### Example 3

### Synthesis of 1,3-bis(2,6-diisopropylphenyl)-2,4-diphenyl-imidazolium (Compound 2) (HBr₂⁻)

The same procedure that was used in Example 2 for the tetrafluoroborate imidazolium salt Compound 1 (BF₄) was used in Example 3, but HBr (48% in water) was used instead of HBF₄.OEt₂. Quantities were the following: N,N′-bis(2,6-diisopropylphenyl)N-(2-oxo-2-phenylethyl)benzimidamide (2.1 g, 3.7 mmol), HBr 48% (2 mL, 17 mmol). 1 (HBr²) was isolated as a white solid (2.6 g, 100% yield). m.p. 207-209° C.; HRMS: m/z calculated for C₃₉H₄₅N₂ 541.3577. found 541.3583. Recrystallization from dichloromethanelhexane at room temperature afforded a few single crystals of Compound 2 (Br).

Crystal structure determination of Compound 2: The Bruker X8-APEX (APEX 2 version 5.1, Bruker (2009). Bruker AXS Inc., Madison, Wis., U.S.A.) X-ray diffraction instrument with Mo-radiation was used for data collection. All data frames were collected at low temperatures (T=100 K) using an 0), Φ-scan mode (0.3° co-scan width, hemisphere of reflections) and integrated using a Bruker SAINTPLUS software package (Bruker (2009). SAINT version V7.60A. Bruker AXS Inc., Madison, Wis., U.S.A.). The intensity data were corrected for Lorentzian polarization. Absorption corrections were performed using the SADABS program (Bruker (2008). SADABS version 2008/1. Bruker Analytical X-Ray System, Inc., Madison, Wis., U.S.A.). The SIR97 (Altomare, A., Buria, M. C., Camalli, M. Cascarano, G. Giacovazzo, C., Guagliardi, A.; Molitemi, A. G. G.; Polidori, G. Spagan, R. SIR 97 (1999) J. Appl. Cryst. 32, 115-122.) software was used for direct methods solution and phase determination, and Bruker SHELXTL\ (Bruker (2003). SHELXTL Software Version 6.14, Dec, Bruker Analytical X-Ray System, Inc., Madison, Wis., U.S.A.) for structure refinement and difference Fourier maps. Atomic coordinates, isotropic and anisotropic displacement parameters of all the nonhydrogen atoms of three compounds were refined by means of a full matrix least-squares procedure on F2.

Crystal structure parameters of Compound 2: size 0.28×0.10×0.09 mm3, monoclinic, space group P 2(1)/n, a=11.983(2) A, b=25.022(4) A, c=11.084(2) A, α=γ=90.0°, β=98.995(2)°, V=3578.7(11) Å³, ρ_{calc}=1.154 g/cm³, Mo-radiation (λ=0.71073 A), T=100(2) K, reflections collected=19901, independent reflections=5163 (Rᵢₙₜ=0.0587), absorption coefficient μ=1.175 mm⁻¹; max/min transmission=0.9016 and 0.7343, 391 parameters were refined and converged at R1=0.0463, wR2=0.1080, with intensity 1>2σ(I), the final difference map was 1.124 and −0.454 e·A⁻³.

### Example 4

### Synthesis of 1,3-bis(2,6-diisopropylphenyl)-2,4-diphenyl-imidazolium (Compound 3) (HCl₂⁻)

The same procedure that was used in Example 1 for the tetrafluoroborate imidazolium salt Compound 1 (BF₄) was used in Example 4, but HCl (12.1M in water) was used instead of HBF₄.OEt₂. Quantities were the following: N,N′-bis(2,6-diisopropylphenyl)-N-(2-oxo-2-phenylethyl)benzimidamide (1 g, 1.9 mmol), HCl (1 mL, 12 mmol). Compound 3 (HCl₂⁻) was obtained as a white solid (1.1 g, 95% yield). m.p. 189-191° C.

### Example 5

### Synthesis of the abnormal N-heterocyclic carbene lithium adduct (Compound 4)

THF (4 mL) was added at −78° C. to a mixture of imidazolium salt Compound 1 (HCl₂⁻) (341 mg, 0.56 mmol) and lithium diisopropylamide (119 mg, 1.11 mmol). After 30 min at −78° C., the mixture was warmed to room temperature and stirred during 2 hours. Solvent was evaporated under vacuum and the residue was extracted with hexane (3×20 mL). Removal of the solvent under vacuum afforded adduct, Compound 4, as a green oil (75 mg, 23% yield). ¹H NMR(C₆D₆, 25° C., 400 MHz): 7.92 (d, J=8.4Hz, 2H, CHₐᵣ), 7.17-6.87 (m, 12H, CHₐᵣ), 6.61-6.60 (m, 2H, CHₐᵣ), 3.18 (sept, J=6.4Hz, 2H, CHCH₃), 2.88 (sept, J=6.4Hz, 2H, CHCH₃), 1.35 (d, J=6.4Hz, 6H, CH₃), 0.97 (d, J=6.4Hz, 6H, CH₃), 0.80 (d, J=6.4Hz, 6H, CH₃), 0.74 (d, J=6.4Hz, 6H, CH₃); ¹³C NMR (C₆D₆, 25° C., 100 MHz): ¹³C NMR (THF, 25° C., 125 MHz): 190 (br, CLi), 144.6 (C), 144.3 (C), 144.1 (C), 140.9 (C), 138.2 (C), 134.1 (C), 132.4 (C), 130.0 (CHₐᵣ), 129.0 (CHₐᵣ)′ 128.7 (CHₐᵣ)′ 128.5 (CHₐᵣ)′ 128.3 (CHₐᵣ)′ 127.4 (CHₐᵣ)′ 127.0 (CHₐᵣ)′ 125.3 (CHₐᵣ)′ 124.6 (CHₐᵣ)′ 123.3 (CHₐᵣ)′ 28.2 (CHCH₃), 27.9 (CHCH₃), 24.2 (CH₃), 22.8 (CH₃), 22.5 (CH₃), 21.6 (CH₃).

### Example 6

### Synthesis of 1-(2,6-diisopropylphenyl)-5-isopropyl-9,9-dimethyl-2,9a-diphenyl-9,9a-diphenyl-dihydro-1H-imidazo[1,2-a]indole (Compound 5)

THF (6 mL) was added at −78° C. to a mixture of imidazolium salt Compound 1 (HBO (580 mg, 0.82 mmol) and lithium diisopropylamide (170 mg, 1.6 mmol). After 30 min at −78° C., the mixture was warmed to room temperature and stirred during 2 hours. The solvent was evaporated under vacuum, and the residue dissolved in 10 mL of Et₂O. 12-Crown-4 (0.26 mL) was added dropwise, and a white precipitated appeared immediately. After filtration, the ether solution was concentrated under vacuum affording a brown oil (200 mg, 45% yield). Colorless single crystals of 3 were obtained from a concentrated Et₂O/hexane solution at −20° C. ¹H NMR (C₆D₆, 25° C., 400 MHz): 7.09-6.63 (m, 16H, CHₐᵣ), 6.39 (s, 1H, CH_{imidazolium}), 4.32 (sept, J=6.8 Hz, 1H, CHCH₃), 3.51 (sept, J=6.8 Hz, 2H, CHCH₃), 1.94 (s, 3H, CH₃), 1.43 (d, J=6.8 Hz, 3H, CH₃), 1.32 (d, J=7.2Hz, 3H, CH₃), 1.26 (d, J=6.4Hz, 3H, CH₃), 1.22 (d, J=6.4Hz, 3H, CH₃), 1.03-1.02 (m, 6H, CH₃), 0.15 (d, J=6.4Hz, 3H, CH₃); ¹³C NMR (C₆D₆, 25° C., 100 MHz): 152.3 (C), 150.8 (C), 144.9 (C), 143.4 (C), 141.0 (C), 137.7 (C), 137.7 (C), 136.5 (C), 133.6 (C), 128.9 (CHₐᵣ)′ 128.5 (CHₐᵣ)′ 127.1 (CHₐᵣ)′ 126.9 (CHₐᵣ)′ 126.2 (CHₐᵣ)′ 126.0 (CH)′ 125.4 (CHₐᵣ)′ 124.3 (CHₐᵣ)′ 121.4 (CHₐᵣ)′ 121.1 (CHₐᵣ)′ 104.1 (NCN), 55.2 (C), 30.6 (CH₃), 29.0 (CHCH₃), 28.5 (CHCH₃), 28.2 (CHCH₃), 25.7 (CH₃), 25.3 (CH₃), 25.2 (CH₃), 24.7 (CH₃), 24.4 (CH₃), 24.0 (CH₃).

Crystal structure parameters of Compound 5: size 0.58×0.54×0.50 mm3, monoclinic, space group P2(1)/c, a=9.3873(2) Å, b=19.0624(4) Å, c=17.7135(4) Å, α=90 °, β=101.0750(10)° γ=90°, V=3110.70(12) Å³, ρ_{calc}=1.155 g/cm³, Mo-radiation (λ=0.71073 A), T=100(2) K, reflections collected=39957, independent reflections=10399 (Rᵢₙₜ=0.0162), absorption coefficient μ=0.066 mm⁻¹; max/min transmission=0.9676 and 0.9626, 503 parameters were refined and converged at R1=0.0427, wR2=0.1150, with intensity 1>2σ (I), the final difference map was 0.549 and −0.206 e·A⁻³.

### Example 7

### Synthesis of Free abnormal N-heterocyclic carbene (Compound 6)

Imidazolium salt Compound 3 (HCl₂) (608 mg, 0.99 mmol) and potassium bis(trimethylsilyl)amide (395 mg, 1.98 mmol) were dissolved in THF at −78° C. and stirred during 30 minutes. The mixture was then warmed to room temperature and stirred during 2 hours. The solvent was removed under vacuum, and the residue extracted with hexane (2×20 mL). After removal of the solvent under vacuum, a green powder was obtained (188 mg, 35% yield). Single yellow crystals of Compound 6 were grown from hexane at −78° C. aNHC Compound 6 is stable at room temperature for a few days under a strict argon atmosphere. M.P. 65° C., decomp.; ¹H NMR (THFd8, 25° C., 400 MHz): 7.53-7.47 (m, 3H, CHₐᵣ), 7.35-7.26 (m, 3H, CHₐᵣ), 7.24-7.00 (m, 8H, CHₐᵣ), 6.98-6.83 (m, 2H, CHₐᵣ), 2.96 (sept, J=6.8 Hz, 2H, CHCH₃), 2.74 (sept, J=6.8 Hz, 2H, CHCH₃), 1.26 (d, J=6.8 Hz, 6 H, CH₃), 1.00 (d, J=6.8 Hz, 6H, CH₃), 0.87 (d, J=6.8 Hz, 6H, CH₃), 0.83 (d, J=6.8 Hz, 6 H, CH₃); ¹³C NMR (THFd8, 25° C., 75 MHz): 201.9 (C_{carbene})′ 146.1 (C), 145.8 (C), 145.5 (C), 141.4 (C), 141.3 (C), 140.3 (C), 136.6 (C), 134.4 (C), 131.3 (CHₐᵣ)′ 130.2 (CHₐᵣ)′ 129.8 (CHₐᵣ)′ 129.4 (CHₐᵣ)′ 128.8 (CHₐᵣ)′ 128.5 (CHₐᵣ)′ 128.2 (CHₐᵣ)′ 126.3 (CHₐᵣ)′ 126.1 (CHₐᵣ)′ 124.4 (CHₐᵣ)′ 29.7 (CHCH₃), 29.4 (CHCH₃), 25.7 (CH₃), 24.3 (CH₃), 23.9 (CH₃), 22.8 (CH₃).

Crystal structure parameters of Compound 6: size 0.38×0.27×0.18 mm3, triclinic, space group P-1, a=12.768(3) Å, b=16.710(4) Å, c=17.768(7) Å, u=106.074(4)°, β=102.608(4)° γ=105.717(3)°, V=3325.6(16) Å³, ρ_{calc}=1.123 g/cm³, Mo-radiation (λ=0.71073 Å), T=100(2) K, reflections collected=35287, independent reflections=11836 (Rᵢₙₜ=0.0515), absorption coefficient μ=0.064 mm⁻¹; max/min transmission=0.9885 and 0.9760, 784 parameters were refined and converged at R1=0.0476, wR2=0.1043, with intensity 1>2σ (1), the final difference map was 0.401 and −0.256 e·AÅ⁻³

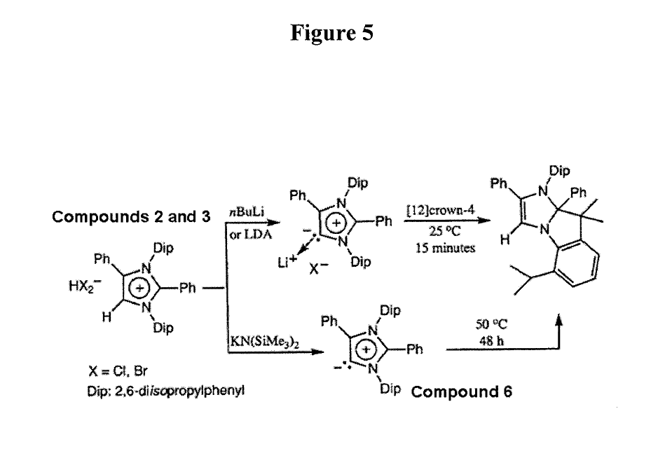

Compound 6 is sensitive to air and quantitatively rearranges as shown in FIG. 5 upon heating in benzene at 50° C. for 48 hours. Compound 6 is stable at room temperature for a few days in both the solid state and in solution. The addition of LiBr or [12]crown-4 to a benzene solution of Compound 6 does not catalyze the rearrangement depicted in FIG. 5. In contrast, the addition of both LiBr and [12]crown-4 induces the rearrangement depicted in FIG. 5 at room temperature, suggesting that the free bromide anion facilitates the proton transfer, through hydrogen bonding.

### Example 8

### Isolation of a C5-Deprotonated Imidazolium, a Crystalline ‘Abnormal’ N-Heterocyclic Carbene

As noted in the detailed description of the methods of making and using the compounds having the structure of Formulas I, II, III, or IV, the publication, E. Aldeco-Perez, “Isolation of a C5-Deprotonated Imidazolium, a Crystalline ‘Abnormal’ N-Heterocyclic Carbene, Science, 326, 556, (2009), and all its supporting online material available at www.sciencemag.org/cgi/content/full/326/5952/556/DC1, is incorporated herein by reference in its entirety.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A stable C5-deprotonated imidazolium carbene compound having the structure of Formula I: 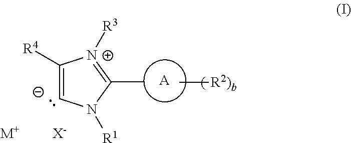
wherein,
R¹, R³, and R⁴ are independently selected from the group consisting of optionally substituted C₁-C₁₀ alkyl, optionally substituted C₂-C₁₀ alkenyl, optionally substituted C₂-C₁₀ alkynyl, optionally substituted C₃-C₁₀ cycloalkyl, optionally substituted C₃-C₁₀ heterocycloalkyl, optionally substituted aryl, and optionally substituted heteroaryl;
R² is, in each instance, independently selected from the group consisting of hydrogen, optionally substituted C₁-C₆ alkyl, optionally substituted C₂-C₆ alkenyl, optionally substituted C₂-C₆ alkynyl, halogen, and hydroxyl;
Ring A is selected from the group consisting of aryl and heteroaryl;
M is either absent or is an alkali metal cation selected from the group consisting of lithium, sodium, potassium, rubidium, and cesium;
X is either absent or is an anion selected from the group consisting of fluoro, chloro, bromo, iodo, trifluoromethanesulfonate, chlorate, acetate, cyanide, thiocynate, oxalate, tetrafluoroborate, nitrate, nitrite, sulfate, sulfite, phosphate, and carboxylate; and
subscript b is an integer of from 0 to 10.

2. The compound of claim 1, wherein ring A is selected from the group consisting of phenyl, benzyl, naphthyl, phenanthrenyl, anthracyl, pyridyl, indolyl, indazolyl, quinoxalinyl, quinolinyl, isoquinolinyl, benzothienyl, benzofuranyl, furanyl, pyrrolyl, thiazolyl, benzothiazolyl, oxazolyl, isoxazolyl, triazolyl, tetrazolyl, pyrazolyl, imidazolyl, and thienyl.

3. The compound of claim 1, wherein R¹, R³, and R⁴ are each optionally substituted phenyl.

4. The compound of claim 1 having the structure of Formula II: 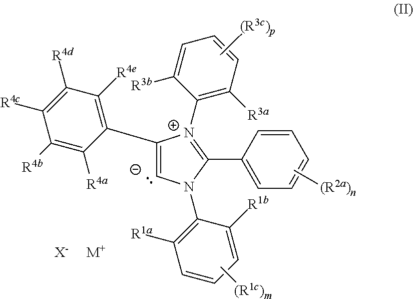
wherein
R^{la}, R^{1b}, R^{3a}, and R^{3b}, are independently selected from the group consisting of hydrogen and optionally substituted C₁-C₁₀ alkyl;
R^{1c}, R^{2a}, R^{1c} are, in each instance, independently selected from the group consisting of hydrogen, optionally substituted C₁-C₆ alkyl, optionally substituted C₂-C₆ alkenyl, optionally substituted C₂-C₆ alkynyl, halogen, and hydroxyl;
R^{4a}, R^{4b}, R^{4c}, R^{4d}, and R^{4e} are independently selected from the group consisting of hydrogen, optionally substituted C₁-C₆ alkyl, optionally substituted C₂-C₆ alkenyl, optionally substituted C₂-C₆ alkynyl, halogen, and hydroxyl;
M is either absent or is an alkali metal cation selected from the group consisting of lithium, sodium, potassium, rubidium, and cesium;
X is either absent or is a halogen anion selected from the group consisting of fluoro, chloro, bromo, and iodo;
subscripts m and p are independently integers of from 0 to 3;
the subscript n is an integer of from 0 to 5; and
salts thereof.

5. A compound of claim 4, wherein R^{1a}, R^{1b}, R^{3a}, and R^{3b} are independently selected from an optionally substituted C₂-C₆ alkyl.

6. A compound of claim 4, wherein R^{1a}, R^{1b}, R^{3a}, and R^{3b} are independently selected from an optionally substituted C₃-C₅ alkyl.

7. A compound of claim 4, wherein R^{1a}, R^{1b}, R^{3a}, and R^{3b} are each isopropyl.

8. A coordination complex comprising:
a metal atom; and
at least one ligand selected from a compound of claim 1.

9. The complex of claim 8, wherein the metal atom is selected from the group consisting of Li, Na, K, Rb, Cs, Be, Mg, Ca, Sr, Ba, Ra, Sc, Y, La, Ti, Zr, Hf, V, Nb, Ta, Cr, Mo, W, Mn, Tc, Re, Fe, Ru, Os, Co, Rh, Ir, Ni, Pd, Pt, Cu, Ag, Au, Zn, Cd, Hg, Al, Ga, In, Tl, Ge, Sn, Pb, Sb, Bi, and Po.

10. The complex of claim 8, further comprising at least one ligand selected from the group consisting of halide, pseudohalide, tetraphenylborate, perhalogenated tetraphenylborate, tetrahaloborate, hexahalophosphate, hexahaloantimonate, trihalomethanesulfonate, alkoxide, carboxylate, tetrahaloaluminate, tetracarbonylcobaltate, hexahaloferrate(III), tetrahaloferrate(III), tetrahalopalladate(II), alkylsulfonate, arylsulfonate, perchlorate, cyanide, thiocyanate, cyanate, isocyanate, isothiocyanate, amines, imines, phosphines, phosphites, carbonyl compounds, alkenyl compounds, allyl compounds, carboxyl compounds, nitriles, alcohols, ethers, thiols and thioethers.

11. (canceled)

12. A reaction mixture comprising a complex of claim 8 under conditions sufficient for catalysis to occur, a solvent and an olefin substrate, wherein said olefin substrate is selected to participate in an olefin metathesis reaction.

13. The reaction mixture of claim 12, wherein said olefin substrate is selected as a substrate for ring closing metathesis.

14. The reaction mixture of claim 12, wherein said olefin substrate is selected as a substrate for ring opening polymerization metathesis.

15. The reaction mixture of claim 12, wherein said olefin substrate is selected as a substrate for cross metathesis.

16. The reaction mixture of claim 12, wherein said olefin substrate is selected as a substrate for acyclic diene polymerization metathesis.

17. A method of making a isolable, stable carbene compound of Formula I, the method comprising:
contacting an imidazolium salt in a solvent with a Brönsted base at a temperature of from about −20 to −100° C.;
warming and stirring the mixture of an imidazolium salt in a solvent with a Brönsted base to room temperature;
removing the solvent under vacuum; and
extracting the product with an extracting solvent.

18. The method of claim 17, wherein the imidazolium salt has the structure of Formula III: 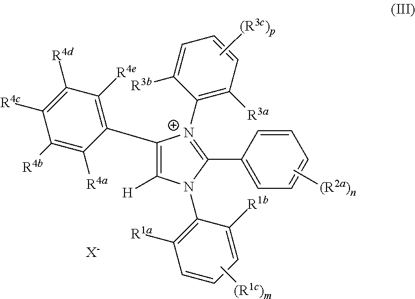
wherein
R^{1a}, R^{1b}, R^{3a}, and R^{3b}, are independently selected from the group consisting of hydrogen and optionally substituted C₁-C₁₀ alkyl;
R^{1c}, R^{2a}, R^{3c} are, in each instance, independently selected from the group consisting of hydrogen, optionally substituted C₁-C₆ alkyl, optionally substituted C₂-C₆ alkenyl, optionally substituted C₂-C₆ alkynyl, halogen, and hydroxyl;
R^{4a}, R^{4b}, R^{4c}, R^{4d}, and R^{4e} are independently selected from the group consisting of hydrogen, optionally substituted C₁-C₆ alkyl, optionally substituted C₂-C₆ alkenyl, optionally substituted C₂-C₆ alkynyl, halogen, and hydroxyl;
X is either absent or is a halogen anion selected from the group consisting of fluoro, chloro, bromo, and iodo;
subscripts m and p are independently integers of from 0 to 3; and
the subscript n is an integer of from 0 to 5.

19. The method of claim 17, wherein the Brönsted base is selected from the group consisting of lithium diisopropylamide, potassium bis(trimethylsilyl)amide, and potassium hexamethyldisilazide.

20. The method of claim 17, wherein the contacting of an imidazolium salt in a solvent with a Brönsted base occurs at a temperature of approximately −78° C.

21. The method of claim 17, wherein the solvent is selected from tetrahydrofuran.

22. The method of claim 17, wherein the extracting solvent is selected from the group consisting of hexane, diethyl ether, and combinations thereof.

23. A method of catalyzing an α-arylation reaction, comprising combining α-arylation reactants with a complex of claim 8 under conditions sufficient for catalysis to occur.

24. A method of catalyzing a Suzuki coupling reaction, comprising combining Suzuki coupling reactants with a complex of claim 8 under conditions sufficient for catalysis to occur.

25. A method of catalyzing an amine arylation reaction, comprising combining amine arylation reactants with a complex of claim 8 under conditions sufficient for catalysis to occur.

26. A method for conducting olefin metathesis, comprising contacting an olefin substrate with a complex of claim 8, under metathesis conditions.

27. The method of claim 26, wherein said olefin substrate is selected as a substrate for ring closing metathesis.

28. The method of claim 26, wherein said olefin substrate is selected as a substrate for ring opening polymerization metathesis.

29. The method of claim 26, wherein said olefin substrate is selected as a substrate for cross metathesis.

30. The method of claim 26, wherein said olefin substrate is selected as a substrate for acyclic diene polymerization metathesis.

31. A method of conducting a reaction selected from the group consisting of a carbon-carbon coupling reaction, a carbon-heteroatom coupling reaction and a 1,2 addition to a multiple bond, said method comprising contacting suitable substrates selected to undergo at least one of said reactions with a complex of claim 8, under suitable reaction conditions.

32. The method of claim 31, wherein said reaction is a carbon-carbon coupling reaction and said suitable conditions include an organic solvent and a temperature of from −30° C. to 190° C.

33. The method of claim 31, wherein said reaction is a carbon-heteroatom coupling reaction and said suitable conditions include an organic solvent and a temperature of from −30° C. to 190° C.

34. The method of claim 31, wherein said reaction is a 1,2-addition to a multiple bond and said suitable conditions include an organic solvent and a temperature of from −30° C. to 190° C.
